(19) United States
(12) Patent Application Publication
(10) Pub. No.: US 2011/0257724 A1
(43) Pub. Date: Oct. 20, 2011

(54) Flexible Stent Device with Magnetic Connections

(75) Inventor: John Kantor, Santa Rosa, CA (US)

(73) Assignee: Medtronic Vascular, Inc., Santa Rosa, CA (US)

(21) Appl. No.: 12/760,615

(22) Filed: Apr. 15, 2010

(51) Int. Cl. A61F 2/82

(52) U.S. Cl. 623/1.15

(57) ABSTRACT

A stent includes a plurality of bands aligned generally along a common longitudinal axis. The plurality of bands includes at least a first band having a plurality of first crowns and a second band adjacent to the first band and having a plurality of second crowns. A magnetic connection joins at least one of the first crowns and at least one of the second crowns.

### FIELD OF THE INVENTION

The present invention is directed to intraluminal stents for use in maintaining open collapsed lumen walls, the intraluminal stent utilizing magnetic connections between adjacent cylindrical elements for improved flexibility for tracking around bends of vessels.

### BACKGROUND OF THE INVENTION

A wide range of medical treatments have been previously developed using “endoluminal prostheses,” which terms are herein intended to mean medical devices which are adapted for temporary or permanent implantation within a body lumen, including both naturally occurring or artificially made lumens. Examples of lumens in which endoluminal prostheses may be implanted include, without limitation: arteries, such as those located within the coronary, mesentery, peripheral, or cerebral vasculature; veins; gastrointestinal tract; biliary tract; urethra; trachea; hepatic shunts; and fallopian tubes. Various types of endoluminal prostheses have also been developed, each providing a uniquely beneficial structure to modify the mechanics of the targeted luminal wall.

For example, stent prostheses have been previously disclosed for implantation within body lumens. Various stent designs have been previously disclosed for providing artificial radial support to the wall tissue, which forms the various lumens within the body, and often more specifically within the blood vessels of the body.

Cardiovascular disease, including atherosclerosis, is the leading cause of death in the U.S. The medical community has developed a number of methods and devices for treating coronary heart disease, some of which are specifically designed to treat the complications resulting from atherosclerosis and other forms of coronary arterial narrowing. One method for treating atherosclerosis and other forms of coronary narrowing is percutaneous transluminal coronary angioplasty, commonly referred to as “angioplasty,” “PTA” or “PTCA.” The objective in balloon angioplasty is to enlarge the lumen of the affected coronary artery by radial hydraulic expansion. The procedure is accomplished by inflating a balloon of a balloon catheter within the narrowed lumen of the coronary artery. In some instances the vessel restenoses chronically, or closes down acutely, negating the positive effects of the angioplasty procedure.

To provide radial support to the treated vessel in order to prolong the positive effects of PTCA, a stent may be implanted in conjunction with the procedure. Effectively, the stent overcomes the tendency of the expanded vessel walls of some patients to close back down, thereby maintaining a more normal flow of blood through that vessel than would be possible if the stent were not in place. Under this procedure, the stent may be collapsed to an insertion diameter and inserted into a body lumen at a site remote from the diseased vessel. The stent may then be delivered to the desired site of treatment within the affected lumen and deployed to its desired diameter for treatment.

Access to a treatment site is most often reached by first entering the femoral artery. A flexible guiding catheter is inserted through a sheath into the femoral artery. The guiding catheter is advanced through the femoral artery into the iliac artery and into the ascending aorta. Further advancement of the flexible catheter involves passage through the aortic arch to allow the guiding catheter to descend into the aortic root where entry may be gained to either the left or the right coronary artery, as desired. To reach some treatment sites, the device must be guided through potentially tortuous and small caliber conduits of the body lumen. Therefore, the stent must be capable of being reduced to a small insertion diameter and must be flexible.

An example of a flexible stent is available from the assignee of the present invention, Medtronic Vascular, Inc., and is known as the S7 stent (shown generally as stent 101 in FIG. 1). The S7 stent has several cylindrical segments, in this case sinusoidally shaped segments 102, which are welded together at the apices or crowns 104 of adjacent segments. FIG. 1 shows stent 101 crimped onto an expandable balloon 106. Alternatively, the stent may be made of an elastic material such that it is positioned in a compressed state and is released to naturally expand within a body lumen. The shape of the sinusoidal segments is described in U.S. Pat. No. 6,344,053 to Boneau, the disclosure of which is incorporated herein by reference in its entirety.

However, stents come in a variety of shapes and sizes. For example, stents formed from a helical winding of wire are useful for defining the cylindrical walls of a stent while being flexible. An example of a helical winding can be found in U.S. Pat. No. 4,886,062 to Wiktor, the disclosure of which is incorporated herein by reference in its entirety. FIG. 2 shows a stent 107 having a wire formed into a series of spiral or helical windings 108, in this case sinusoidally shaped helical windings 108. The adjacent helical windings 108 together form a cylindrical body of stent 107. Just as in FIG. 1, FIG. 2 shows stent 107 on an expandable balloon 110. However, stent 107 could be a self-expanding stent, such that when released within a body lumen, it naturally expands.

In another example, U.S. Pat. No. 6,565,599 to Hong et al., the disclosure of which is incorporated herein by reference in its entirety, describes bands formed from sinusoidally shaped segments that are interconnected by elongated struts of a flexible polymer material, which hold the bands or rows apart from one another. U.S. Pat. No. 6,475,237 to Drasler et al., the disclosure of which is incorporated herein by reference in its entirety, describes a strut wherein a portion thereof is made thinner and more flexible such that the strut can flex at those locations.

U.S. Pat. No. 5,035,706 to Gianturco, the disclosure of which is incorporated herein by reference in its entirety, describes the use of interlocking rings to connect adjacent segments. U.S. Pat. No. 6,387,122 to Cragg, the disclosure of which is incorporated herein by reference in its entirety, describes a helical stent in which subsequent windings are connected by loop members made from sutures, staples or rings of metal or plastic. Connecting rows of a helical stent, provides more contact between the stent and the lumen walls (i.e., more scaffolding) and thus provides better support for the lumen wall.

The different types of connecting elements discussed above for connecting adjacent cylindrical segments or windings may require a compromise between stent coverage or scaffolding by the stent at the treatment site when the stent is deployed and flexibility of the stent during delivery to and implantation of the stent at the treatment site. Elongated connecting elements, for example, may provide increased flexibility over having cylindrical segments that are welded directly to each other. However, elongated connecting elements may separate the segments, providing less scaffolding by the stent at the treatment site. It is desirable to maximize both flexibility and scaffolding in a stent.

### BRIEF SUMMARY OF THE INVENTION

The present invention is directed to an intraluminal stent device having at least two bands with magnetic connections connecting them to provide a flexible stent. In one embodiment, a stent includes a plurality of bands aligned generally along a common longitudinal axis. The plurality of bands includes at least a first band having a plurality of first crowns and a second band adjacent to the first band and having a plurality of second crowns. A magnetic connection connects at least one of the first crowns with at least one of the second crowns.

In another embodiment, the magnetic connections between bands of a stent are separable such that during advancement of the stent through a vessel, some of the magnetic connections may temporarily separate such that a gap is formed between magnets of the magnetic connection to permit the stent to bend, for example, around a bend in the vessel.

### BRIEF DESCRIPTION OF DRAWINGS

The foregoing and other features and advantages of the invention will be apparent from the following description of the invention as illustrated in the accompanying drawings. The accompanying drawings, which are incorporated herein and form a part of the specification, further serve to explain the principles of the invention and to enable a person skilled in the pertinent art to make and use the invention. The drawings are not to scale.

FIG. 1 is a front plan view of a prior art stent having bands formed from sinusoidally shaped cylindrical segments.

FIG. 2 is a front plan view of a prior art stent having bands formed from the helical windings of a sinusoidally shaped ribbon or wire.

FIG. 3 illustrates a perspective view of an embodiment of a stent of the present invention.

FIG. 4 illustrates a plan view the stent of FIG. 3 opened and flattened.

FIGS. 5-12 illustrate various embodiments of magnetic connections between crowns of adjacent cylindrical bands of the stent of FIG. 3.

FIG. 13 illustrates the magnetic connection of FIG. 7 with the crowns of adjacent cylindrical elements not aligned.

FIG. 14 illustrates a schematic of the stent of FIG. 3 in the radially compressed configuration during delivery through a relatively straight portion of a vessel.

FIG. 15 illustrates a schematic of the stent of FIG. 3 in the radially compressed configuration during delivery through a curved portion of a vessel.

FIG. 16 illustrates a schematic of the stent of FIG. 3 in the radially compressed configuration during delivery through in a relatively straight portion of a vessel after having passed through the curved portion shown in FIG. 12.

FIG. 17 illustrates another embodiment of a stent of the present invention.

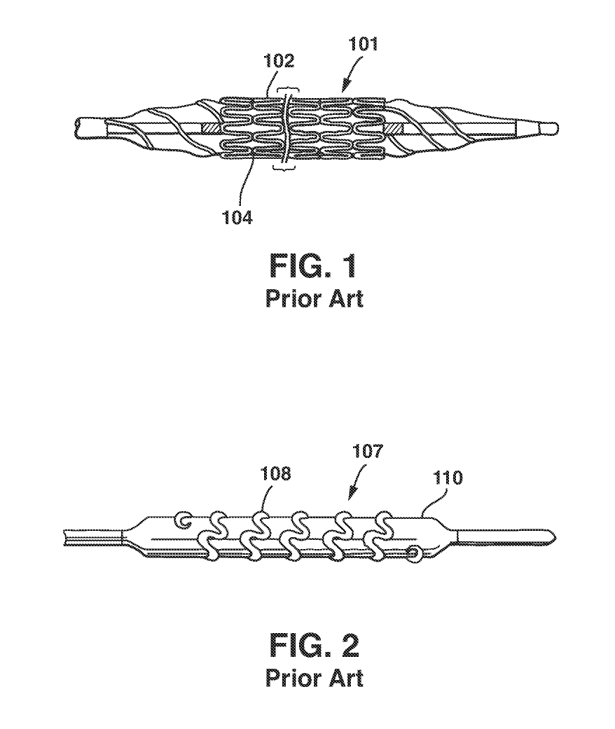

### DETAILED DESCRIPTION OF THE INVENTION

Specific embodiments of the present invention are now described with reference to the figures, wherein like reference numbers indicate identical or functionally similar elements. The terms “distal” and “proximal” are used in the following description with respect to a position or direction relative to the treating clinician. “Distal” or “distally” are a position distant from or in a direction away from the clinician. “Proximal” and “proximally” are a position near or in a direction toward the clinician.

The following detailed description is merely exemplary in nature and is not intended to limit the invention or the application and uses of the invention. Although the description of the invention is in the context of treatment of blood vessels such as the coronary, carotid and renal arteries, the invention may also be used in any other body passageways where it is deemed useful. Furthermore, there is no intention to be bound by any expressed or implied theory presented in the preceding technical field, background, brief summary or the following detailed description.

Further, as used herein, the term “bands” refers generally to cylindrically shaped segments, helical windings, rows, columns, or other similar items or terms known to those of ordinary skill in the stent art.

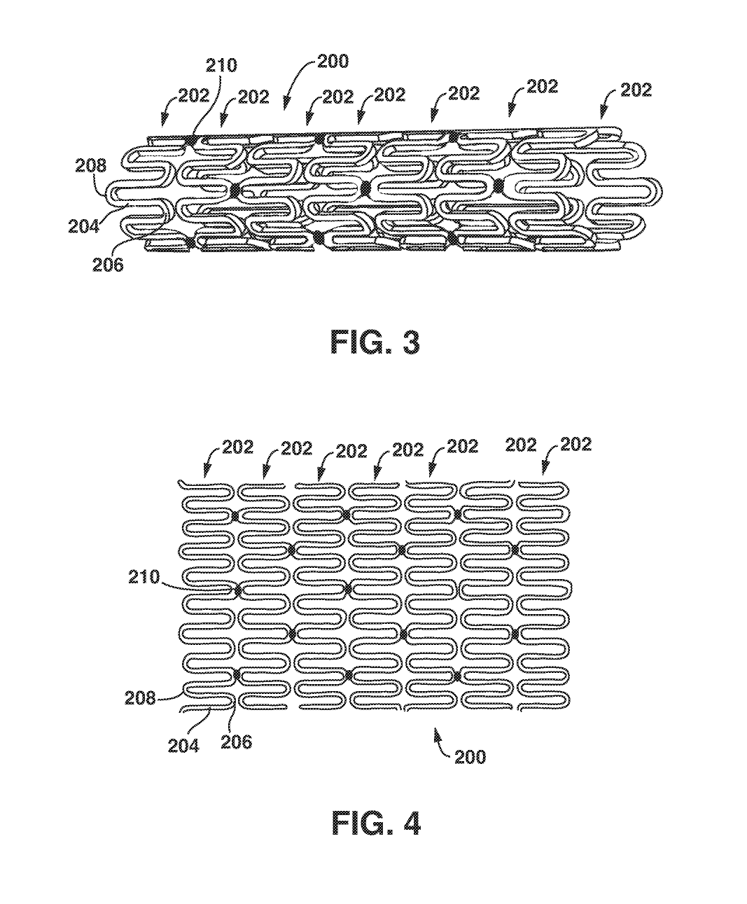

FIG. 3 shows a stent 200, in which the stent body comprises adjacent cylindrical segments or bands 202. In this particular embodiment, bands 202 of stent 200 of FIG. 3 are separate cylindrical segments formed from generally sinusoidally shaped segments including generally longitudinal struts 204 interconnected by crowns 206, 208. Adjacent bands 202 are connected by magnetic connections 210 at crowns 206/208, as described in more detail below. FIG. 4 shows stent 200 cut open along a line parallel to the center axis of the stent and laid flat for illustrative purposes. As shown, a plurality of magnetic connections 210 is provided between adjacent bands 202. However, in the embodiment shown, not every crown 206 is connected to the adjacent crown 208. It would be understood by one of ordinary skill in the art that all or only some of crowns 206 may be connected to crowns 208 of adjacent bands 202. For example, if there are N number of crowns 206 on a first band, there may be between one and N magnetic connections 210 between the first band and an adjacent second band. Further, the number and position of magnetic connections 210 between a first band and an adjacent second band may be different than the number and position of magnetic connections 210 between the second band and an adjacent third band. Further, although the examples discussed herein feature bands that are sinusoidally shaped, they are provided as examples only and are not intended to limit the scope of this invention.

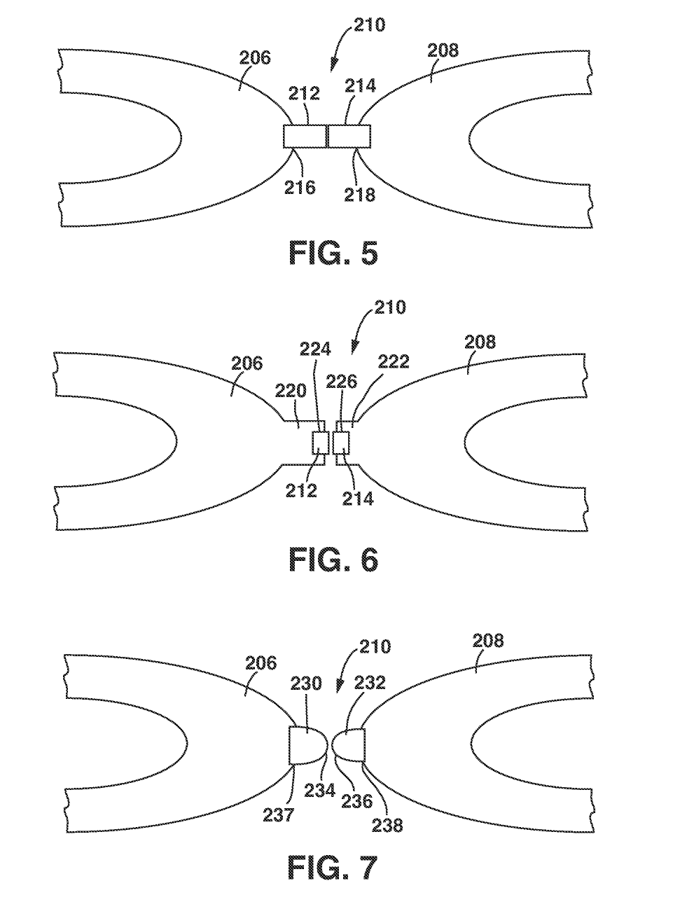

FIG. 5 shows an embodiment of a magnetic connection 210 between adjacent bands of a stent of the present invention, shown between a crown 206 of one band and a crown 208 of an adjacent band for example where the bands are sinusoidally shaped cylindrical segments. The magnetic connection 210 shown in FIG. 5 includes a first magnet 212 coupled to crown 206 of a first band and a second magnet 214 coupled to crown 208 of a second band adjacent to the first band. In the embodiment of FIG. 5, crowns 206, 208 each include a recess 216, 218 into which magnets 212, 214 are inserted, respectively. Magnets 212, 214 may be coupled to crowns 206, 208 using an adhesive, a friction fit, notches, keyway, dovetail groove, tongue and groove, press fit, screw threads, other mechanical connections or welding. In an embodiment (not shown), a through-hole may be drilled in a crown, and a cylindrical magnet can be press-fitted in the hole. As would be understood by those of ordinary skill in the art, opposite poles of the magnets should face each other such that there is an attractive force between the magnets. The magnetic connection 210 between adjacent bands of the stent allows the bands to move angularly relative to each other and/or even temporarily separate from each other when negotiating tortuous paths of the vasculature during delivery of the stent to a desired location. After the stent is deployed at the treatment site, disengaged magnetic connections 210 between adjacent bands of the stent may reconnect to restore adjacent bands to close proximity with each other, as will be explained in more detail below. The attractive force of the magnets permits the magnetic connection 210 to be temporarily broken and restored during delivery of the stent, as will also be explained in more detail below.

FIGS. 6-10 show additional embodiments of magnetic connections 210. It will be understood by one of ordinary skill in the art that these embodiments are not exhaustive, nor are they mutually exclusive. Thus, features of the different embodiments may be mixed and matched without departing from the invention hereof. Further, other variations to the magnetic connections may be made, as would be apparent to those skilled in the art.

FIG. 6 shows an embodiment of a magnetic connection 210 similar to the embodiment of FIG. 5 except that a longitudinal extension 220 is provided in crown 206 and a longitudinal extension 222 is provided in crown 208 of the adjacent band. Longitudinal extensions 220 and 222 extend towards each other. Longitudinal extension 220 includes a recess 224 with magnet 212 disposed at least partially therein. Longitudinal extension 222 includes a recess 226 with magnet 214 disposed at least partially therein.

FIG. 7 shows another embodiment of a magnetic connection 210 similar to the embodiment of FIG. 5 except that magnets 230 and 232 of FIG. 7 each have rounded ends 234, 236. In particular, magnet 230 is at least partially disposed in a recess 237 of crown 206 and extends toward crown 208 of the adjacent band. End 234 of magnet 230 closest to crown 208 is rounded or convex. Similarly, magnet 232 is at least partially disposed in a recess 238 in crown 208 and extends toward crown 206 in the adjacent band. End 236 of magnet 232 closest to crown 206 is rounded or convex.

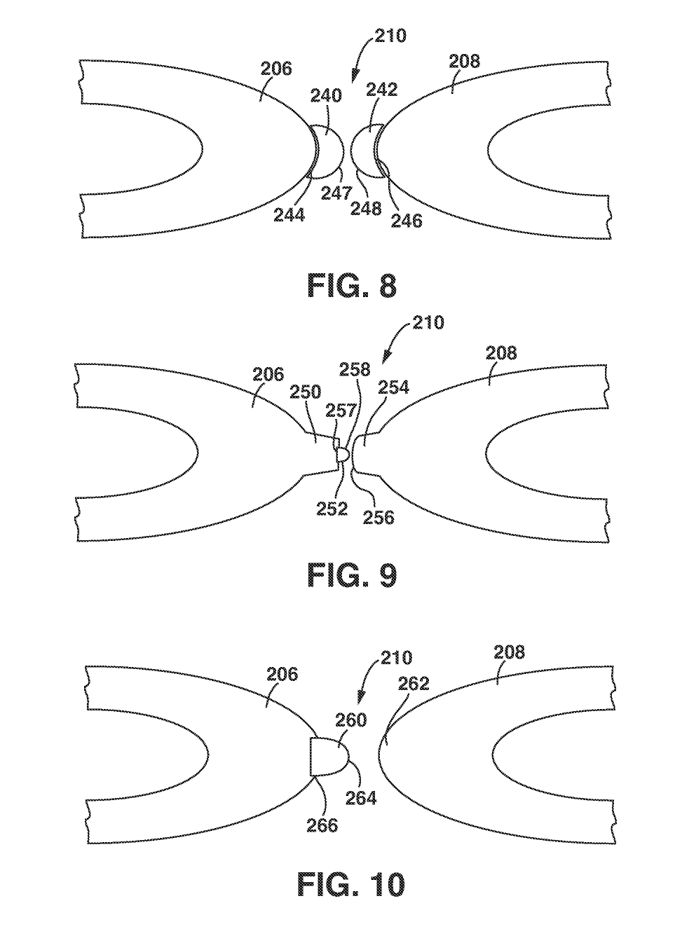

FIG. 8 shows another embodiment of a magnetic connection 210 similar to the embodiment of FIG. 7 except that magnets 240 and 242 of FIG. 8 are coupled to a convex surface of crowns 206 and 208, respectively, instead of being disposed in a recess. Thus, a concave surface 244 of magnet 240 is coupled to a convex surface of crown 206. Magnet 240 may also include a rounded convex surface 247 facing crown 208 of the adjacent band. Similarly, a concave surface 246 of magnet 242 is coupled to a convex surface of crown 208. Magnet 242 may also include a rounded, convex surface 248 facing crown 206 of the adjacent band.

FIG. 9 shows another embodiment of a magnetic connection 210. In the embodiment shown in FIG. 9, crown 206 includes an extension 250 extending towards crown 208 of the adjacent band. A magnet 252 is disposed in a recess 247 of extension 250. Magnet 252 includes a rounded, convex end 258 adjacent crown 208. Crown 208 does not include a magnet in this embodiment. Instead crown 208 includes an extension 254 made from a material that, while not being magnetized, is magnetically attracted to magnet 252. For example, extension 254 may be made from some stainless steel materials, iron, nickel, cobalt, and other materials known to those of ordinary skill in the art. Extension 254 may be made of the same material as the rest of the stent, provided that it is magnetically attracted to magnets 252. Extension 254 in this embodiment includes a rounded, convex end 256 adjacent magnet 252.

FIG. 10 shows another embodiment of a magnetic connection 210 similar to the embodiment of FIG. 7 except that crown 208 does not include a magnet. In particular, a magnet 260 is disposed in a recess 266 in crown 206, and extends towards crown 208 in an adjacent band. Magnet 260 includes a rounded, convex end 264 adjacent crown 208. Crown 208 is made from a material that, while not being magnetized, is magnetically attracted to magnet 252, such as, for example, some stainless steel materials, iron, nickel, cobalt, and other materials known to those of ordinary skill in the art. Crown 208 may be made of the same material as the rest of the stent, provided that it is magnetically attracted to magnets 260. In another embodiment, crown 208 may be made of the same ferromagnetic material as the rest of the stent. Crown 208 may be magnetized by subjecting it to a magnetic field while locally heating crown 208 above its Curie temperature T_{c} and allowing it to cool while in the field.

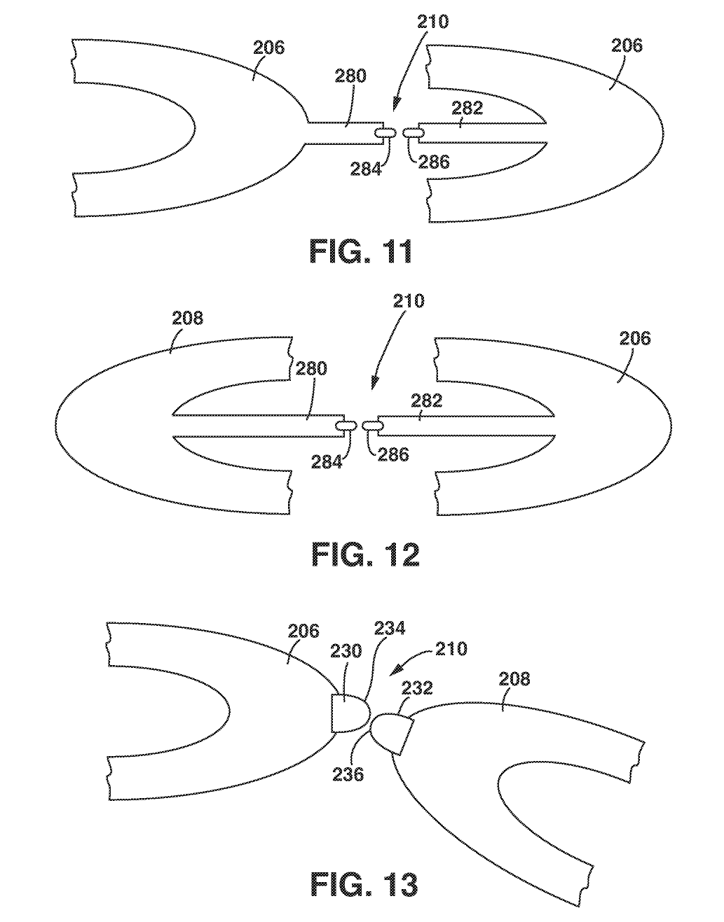

As would be understood by those skilled in the art, the various features described above with respect to FIGS. 5-10 may be interchanged and combined. Further, although the magnetic connections have been described with respect to a “peak-to-peak” connection of adjacent crowns, one skilled in the art would recognize that the magnetic connections may be made with respect to a “peak-to-valley” connection, as shown schematically in FIG. 11, or a “valley-to-valley” connection, as shown schematically in FIG. 12. In particular, extension elements 280, 282, when magnetically joined, may form a strut that bridges the gap between a peak crown and/or a valley crown, with magnets 284, 286 coupled to the ends of extension elements 280, 282, respectively.

FIG. 13 shows an example of the flexibility of a stent having magnetic connections 210. Although FIG. 13 uses the embodiment of FIG. 7, it would be understood by those of ordinary skill in the art that any of the embodiments described herein, or equivalents thereof, may be used to achieve similar stent flexibility. As shown, in FIG. 13, as crown 206 of a band is angularly displaced relative to crown 208 of the adjacent band, such as when the stent is being delivered around a tight curve or bend in the vasculature, magnets 230, 232 may be angularly displaced relative to each other while ends 234, 236 remain magnetically connected to each other. When the stent is further advanced into a straight portion of the vasculature, the magnetic connection 210 may realign to the configuration shown, for example, in FIG. 7.

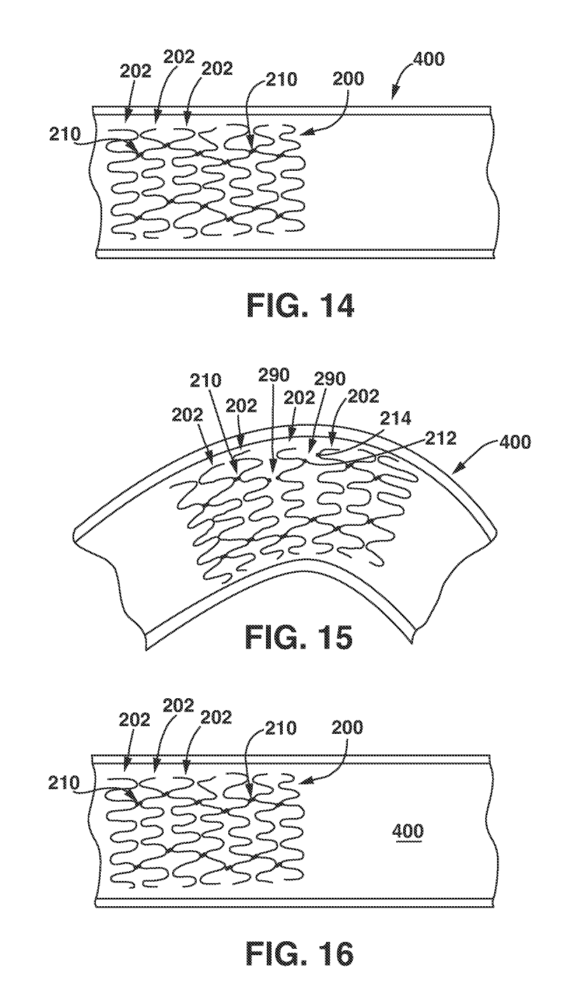

As further shown schematically in FIGS. 14-16, some magnetic connections 210 may be temporarily broken while the stent 200 is advanced around a bend in the vasculature. In particular, as illustrated in FIG. 14, stent 200 is advanced along a relatively straight portion of a vessel 400. For clarity, the delivery system is omitted and stent 200 is shown relatively large, even though it is in a radially compressed configuration for delivery. As illustrated in FIG. 14, stent 200 is in the configuration shown, for example in FIG. 3, with magnetic connections 210 coupling adjacent bands 202 together. As stent 200 is advanced around a bend in the vessel 400, as shown in FIG. 15, magnetic connections 210 along an outer radius of the bend may become separated such that gaps 290 are disposed between magnets 212, 214 of adjacent bands 202. Such gaps 290 may occur at only some of the locations. The magnetic connections 210 at the inner radius of the bend in the vessel 400 may slide or angularly shift without disconnecting, for example, as shown in FIG. 13. When the stent 200 is advanced or retracted to a straight portion of vessel 400, the magnetic connections 210 that were separated may become reconnected due to the magnetic attraction between magnets 212, 214, as shown in FIG. 16. This separation and/or shifting of the magnetic connections 210 prevents the structural elements of the stent from becoming plastically distorted during delivery through the vasculature. Further, although the flexibility of stent 200 with magnetic connections 210 has been described with respect to delivery of the stent in its radially compressed configuration, those of ordinary skill in the art would recognize that such flexibility may also be useful when the stent is in a radially expanded configuration at the treatment site, for example, if the treatment site includes a bend or an irregularity. The magnetic connections 210 may shift as shown in FIG. 13, or may separate as shown in FIG. 15, for the stent 200, in a radially expanded configuration, to conform to such a bend or irregularity (not shown).

In an embodiment, the feature of joining or re-joining magnetic stent crowns to each other in-vivo may be applied to a method of clinically deploying two or more completely un-joined stents of the invention. A plurality of relatively short stents having magnetic crowns at least at mating ends may be joined together in a patient's vessel to create a single, longer stent that might otherwise be difficult to navigate to the implantation site because of its length and vessel tortuosity. Multiple stents having magnetic crowns can be delivered separately on a single catheter or via separate catheterization steps.

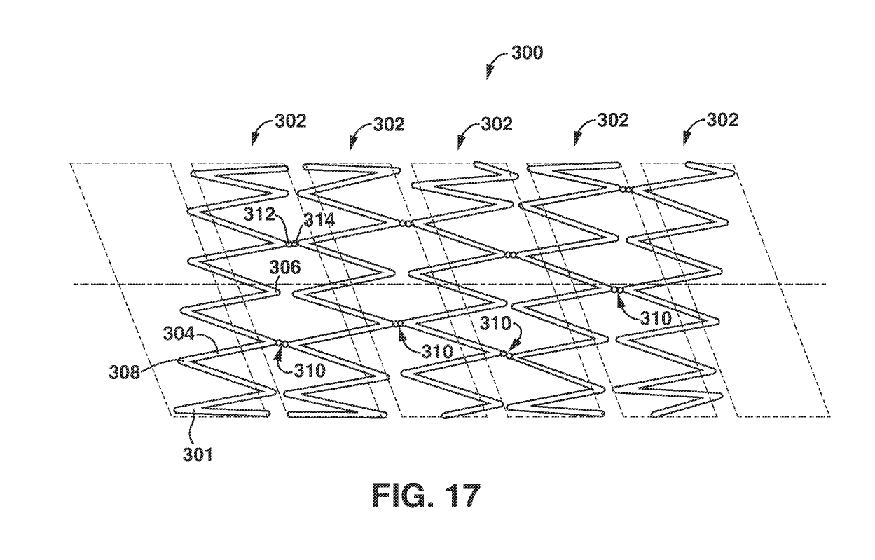

FIG. 17 shows another embodiment of a stent 300. In this embodiment, stent 300 is a helically wound stent. In the embodiment shown, stent 300 is made from a wire 301 which is bent into a sinusoidally shaped waveform having struts 304 and crowns 306, 308. The waveform is helically wound, for example around a mandrel, to form the tubular structure of the stent, as known to those of ordinary skill in the art. The helical winding forms a plurality of bands 302, as shown. Selected crowns 306, 308 of adjacent bands include magnets 312, 314, respectively, forming a magnetic connections 310. As would be understood by those of ordinary skill in the art, various magnet configurations, for example and not by way of limitation, those shown in FIGS. 5-10 above and combinations thereof, may be used in the embodiment of FIG. 17.

The magnets described herein may be made of any suitable magnetic material. For example, and not by way of limitation, the magnets may be permanent magnets, such as samarium-cobalt (SmCo) magnets, neodymium-iron-boron (NeFeB) magnets, aluminum-nickel-cobalt (AlNiCo) magnets, ferrite magnets, platinum-cobalt alloy magnets, and other permanent magnets known to those of ordinary skill in the art. Neodymium-iron-boron magnets are considered to offer the highest energy product per unit mass (which may be useful due to size limitations), and highest energy product per unit cost of any current permanent magnet material. For example, and not by way of limitation, Bob Johnson Associates offers a neodymium-iron-boron magnet under the trade name MICRO-MAGNET™ designed for medical applications. This magnet is offered in with energy products up to 52 MGOe, and is coated with an inert biocompatible protective coating.

Biocompatible protective coatings may be applied to magnets that are not biocompatible. Protective coatings may be, for example and not by way of limitation, PYROLITE® or BIOLITE® pyrolytic carbon, both by Sulzer Carbomedics, Inc., Austin Tex., Parylene chemical vapor deposited poly(p-xylylene) polymers, biocompatible polymeric materials, gold, titanium, or other biocompatible coatings known to those of ordinary skill in the art. The magnets may alternatively be magnetized materials rather than permanent magnets.

The stents described herein may be made of suitable stent materials known to those of ordinary skill in the art. For example, and not by way of limitation, stainless steel, nickel-titanium alloys, magnesium alloys, cobalt-chromium-molybdenum alloys, metal combinations such as drawn-filled-tubing, and other materials known to those of ordinary skill in the art. In an embodiment, magnets that are not biocompatible may be encased within a hollow slender tube that may be formed into a helically wound stent such that the magnets are disposed within the crowns, thus rendering protective coatings unnecessary.

While various embodiments according to the present invention have been described above, it should be understood that they have been presented by way of illustration and example only, and not limitation. It will be apparent to persons skilled in the relevant art that various changes in form and detail can be made therein without departing from the spirit and scope of the invention. Thus, the breadth and scope of the present invention should not be limited by any of the above-described exemplary embodiments, but should be defined only in accordance with the appended claims and their equivalents. It will also be understood that each feature of each embodiment discussed herein, and of each reference cited herein, can be used in combination with the features of any other embodiment. All patents and publications discussed herein are incorporated by reference herein in their entirety.

What is claimed is:

1. A flexible stent comprising:
a plurality of bands aligned generally along a common longitudinal axis, wherein the plurality of bands includes at least a first band having a plurality of first crowns and a second band adjacent to the first band and having a plurality of second crowns; and
a magnetic connection between at least one of the first crowns and at least one of the second crowns.

2. The flexible stent of claim 1, wherein the magnetic connection includes a first magnet coupled to the at least one of the first crowns and a second magnet coupled to the at least one of the second crowns.

3. The flexible stent of claim 2, wherein at least one of the first magnet and the second magnet is a permanent magnet.

4. The flexible stent of claim 2, wherein at least one of the first magnet and the second magnet includes a rounded end closest to the other of the first magnet and the second magnet.

5. The flexible stent of claim 4, wherein the first magnet and the second magnet have rounded ends facing each other.

6. The flexible stent of claim 2, wherein the first magnet is disposed in a recess in the first crown.

7. The flexible stent of claim 2, wherein the first crown includes an extension element and the first magnet is coupled to the extension element.

8. The flexible stent of claim 1, wherein said first band and said second band are formed by cylindrical segments.

9. The flexible stent of claim 1, wherein said bands are formed by windings of a helical stent body.

10. A flexible stent comprising:
a first band;
a second band adjacent said first band; and
a connection between said first and second bands, wherein said connection is separable and reconnectable during delivery of the stent to a treatment site.

11. The flexible stent of claim 10, wherein the connection is a magnetic connection.

12. The flexible stent of claim 10, wherein the first band includes a plurality of first crowns and the second band includes a plurality of second crowns, and wherein the connection is disposed between one of the plurality of first crowns and one of the plurality of second crowns.

13. The flexible stent of claim 10, wherein the stent comprises a plurality of bands, and wherein a separable and reconnectable connection is disposed between each adjacent band in the plurality of bands.

14. The flexible stent of claim 13, wherein the connections are magnetic connections.

15. A method of delivering a stent to a treatment site in a vessel, wherein the stent includes a plurality of a plurality of bands aligned generally along a common longitudinal axis, wherein adjacent bands in the plurality of bands are connected by separable connections, comprising the steps of:
advancing the stent in a radially compressed configuration through a lumen of the vessel;
permitting at least one of the connections to be separated while the stent is being advanced; and
after the connection is separated, permitting the connection to be reconnected.

16. The method of claim 15, wherein the connections are magnetic connections.

17. The method of claim 16, wherein at least one of the magnetic connections separates during advancement through a bend in the vessel.

18. The method of claim 17, wherein the at least one separated magnetic connection is reconnected after the stent has been advanced through the bend.
